(19) United States
(12) Patent Application Publication
(10) Pub. No.: US 2024/0382919 A1
(43) Pub. Date: Nov. 21, 2024

(54) SHORT CONTACT REACTOR, AND SYSTEM AND PROCESS USING THE SAME IN PREPARATION OF ETHYLENE AND PROPYLENE FROM METHANOL

(71) Applicants: CHINA PETROLEUM & CHEMICAL CORPORATION, Beijing (CN); SHANGHAI RESEARCH INSTITUTE OF PETROCHEMICAL TECHNOLOGY, SINOPEC, Shanghai (CN)

(72) Inventors: Xiaohong LI, Shanghai (CN); Guozhen QI, Shanghai (CN); Zhinan YU, Shanghai (CN); Fei PENG, Shanghai (CN); Hongtao WANG, Shanghai (CN); Yijun ZHENG, Shanghai (CN)

(21) Appl. No.: 18/696,433

(22) PCT Filed: Sep. 28, 2022
(86) PCT No.: PCT/CN2022/121966

§ 371 Date: Mar. 28, 2024

(30) Foreign Application Priority Data
Sep. 29, 2021 (CN) 202111150391.1

(51) Int. Cl. B01J 8/12; B01J 8/00; B01J 8/08; C07C 1/20

(52) U.S. Cl. CPC B01J 8/12; B01J 8/0055; B01J 8/085; C07C 1/20; B01J 2208/00548

(57) ABSTRACT

A short contact reaction system for preparing ethylene and propylene from methanol includes an MTO short contact reactor, a riser reactor, a dense bed, and a stripper. The MTO short contact reactor has the following components coaxially distributed from inside to outside: a methanol feeding pipeline, a filter pipe wall, a product gas channel, and a catalyst distributor arranged at the top of the reactor, and a seal pipe arranged at the bottom of the reactor. The seal pipe is located in the stripper. The diameter at the top of the product gas channel is smaller than the diameter at the bottom of the product gas channel. Methanol is in crossflow contact with the descending coked catalyst II in the MTO short contact reactor.

### TECHNICAL FIELD

The present invention relates to a short contact reactor, as well as a system and a reaction process using the reactor to produce ethylene propylene from methanol.

### BACKGROUND ART

Low-carbon olefins, namely ethylene and propylene, are two important basic chemical raw materials, and their demand is constantly increasing. Generally, ethylene and propylene are produced through a route from petroleum. However, due to the limited supply and high price of petroleum resources, the cost of producing ethylene and propylene from petroleum resources continues to increase. The technology of converting alternative raw materials to produce ethylene and propylene has received more and more attention. Among others, an important type of alternative raw materials for the production of low-carbon olefins are oxygen-containing compounds, such as alcohols (methanol, ethanol), ethers (dimethyl ether, methyl ethyl ether), and esters (dimethyl carbonate, methyl formate). These oxygen-containing compounds can be obtained through the conversion of energy sources such as coal, natural gas, and biomass. Some oxygen-containing compounds can already be produced on a large scale, e.g. methanol, which can be produced from coal or natural gas. The production process is very mature and can achieve a production scale of millions of tons. Therefore, in recent years, the process of converting methanol to olefins (MTO) has been greatly developed. Three companies have turned their technologies into industrial applications, and many related technologies exist.

CN102464534B and CN102372538A disclose processes for converting methanol into low carbon olefins in sectors. The methanol enters the lower premixing zone or catalyst mixing tube and the upper main reaction zone for reaction respectively.

CN102276398A discloses a process, in which liquid methanol enters the initial contact zone to exchange heat with the spent catalyst, and then ascends into the main reaction zone for reaction to produce ethylene and propylene. Because the reaction conditions in the premixing zone, the catalyst mixing tube, and the initial contact zone are not suitable for methanol conversion, the above technologies will result in the carbon-based loss of methanol and the lower double-olefins selectivity.

### SUMMARY OF THE INVENTION

One of the technical problems to be solved by the present invention is to provide a short contact reaction system for producing ethylene and propylene from methanol in order to solve the technical problem of low ethylene and propylene selectivity in the prior art. This system has the advantage of high ethylene and propylene selectivity.

In order to achieve the aforementioned objects, according to the first aspect of the present invention, the present invention provides a short contact reactor, which can be used in a reaction system for producing ethylene and propylene from methanol. The short contact reactor comprises the following components coaxially distributed from inside to outside:
a feeding pipeline, having a feeding port at its lower end for introducing a gaseous feedstock, and allowing the feedstock to be transported from bottom to top, wherein feeding pores are distributed on the feeding pipeline, so that the feedstock is transported outward from the feeding pipeline in a substantially radial direction, and preferably, the feeding pipeline has a closed top at its upper end;
an axial-radial reaction space, which is defined by the inner feeding pipeline and an outer filter pipe wall, so that the feedstock transported radially outward contacts the catalyst transported axially from top to bottom in crossflow, and the gas-solid contact time is less than 3 seconds in the reaction space;
a filter pipe wall, which has a certain pore diameter so that the feedstock continues to be transported through pores outward and into a product gas channel, and the catalyst density in the product gas channel is less than 10 kg/m³; and a reactor shell, which defines a product gas channel together with the filter pipe wall, and has such a shape and structure that the residence time of a product gas in the product gas channel is less than 15 seconds;
and the reactor also has a catalyst distributor arranged at its top, which distributor has a certain pore fraction, pore diameter and distribution thereof, so as to transport the catalyst by gravity from top to bottom, and allow the catalyst density in the reaction space to reach the range from 80 kg/m³ to 400 kg/m³.

The short contact reactor according to the present invention can be used as the MTO short contact reactor to configure an MTO short contact reaction system, comprising: an MTO short contact reactor, a riser reactor, a dense bed, and a stripper;
wherein the MTO short contact reactor is used to convert methanol to an olefin-rich product;
the riser reactor is used to convert a mixed light hydrocarbon feed, including a mixture of C₄-C₆ non-aromatic hydrocarbons from the product of the MTO short contact reactor, into an olefin-rich product, which ascends into the dense bed;
the dense bed is used to store and provide a catalyst required by the MTO short contact reactor and convert a by-product oxide feed from the reaction product;
the stripper is used to remove a reaction product entrained by the coked catalyst from the MTO short contact reactor.

Among others, the MTO short contact reactor includes the following components coaxially distributed from inside to outside: a methanol feeding pipeline, a filter pipe wall, a product gas channel, and a catalyst distributor arranged at the top of the reactor, a seal pipe arranged at the bottom of the reactor; the seal pipe is located in the stripper; the diameter at the top of the product gas channel is smaller than the diameter at the bottom of the product gas channel.

According to another aspect of the present invention, the present invention provides a process for producing ethylene and propylene from methanol through a short contact reaction, wherein the process is carried out with the reaction system of the present invention, and the process comprises:
a) rendering a methanol feedstock to enter an MTO short contact reactor via a methanol feeding pipeline, and contact and react with a descending catalyst to produce a methanol reaction product and a coked catalyst I; rendering the methanol reaction product to enter a product gas channel via a filter pipe wall, leave the MTO short contact reactor and merge into a reaction product;
rendering the coked catalyst to descend into a stripper via a seal pipe;
b) rendering a mixed light hydrocarbon feed and a steam to enter a riser reactor, contact and react with a regenerated catalyst, and ascend into a dense bed;
c) rendering a by-product oxide feed to enter the dense bed, and contact and react with a catalyst to produce a reaction product and a coked catalyst II; rendering the coked catalyst II to enter the MTO short contact reactor via a catalyst flow controller and a catalyst distributor;
d) rendering a stripping medium to enter a stripper and contact the catalyst to perform stripping;
rendering the resulting stripped product to merge into the reaction product and the resulting spent catalyst to enter a regenerator for regeneration to produce a regenerated catalyst;
e) rendering the reaction product to enter a subsequent separation system for separation to produce a mixed light hydrocarbon feed and a by-product oxide feed.

According to the process of the present invention, methanol is in crossflow contact with the descending coked catalyst II in the MTO short contact reactor. The reaction by contacting methanol with the catalyst in crossflow under a very short contact time condition can obtain products with high ethylene and propylene selectivity. The special product gas channel can achieve rapid separation of reaction product and catalyst. Unconverted methanol enters the by-product oxide feed through the separation system and continues to be converted in the dense bed. Due to the increased dilution ratio, this part of methanol can also achieve higher ethylene and propylene selectivity. Therefore, using the technical solution of the present invention and using a catalyst such as SAPO-34, the ethylene and propylene carbon-based selectivity can reach 90.4 wt. %, achieving good technical results.

The solution of the present invention better solves the problem of low ethylene and propylene selectivity and can be used in the MTO industrial production.

### DESCRIPTION OF THE DRAWINGS

FIG. 1 is a schematic diagram of a short contact reactor according to a preferred embodiment of the present invention.

FIG. 2 is a schematic diagram of a reaction system according to a preferred embodiment of the present invention.

FIG. 3 is a cross-sectional view of a short contact reactor according to a preferred embodiment of the present invention.

### EXPLANATION OF REFERENCE NUMBERS

1: short contact reactor; 2: riser reactor;
3: dense bed; 4: stripper;
5: regenerated catalyst; 6: spent catalyst;
7: methanol feeding pipeline; 8: filter pipe wall;
9: product gas channel; 10: bottom of the product gas channel;
11: top of the product gas channel; 12: methanol feedstock;
13: mixed light hydrocarbon feed; 14: seal pipe;
15: catalyst distributor; 16: stripping medium;
17: stripped product; 18: methanol reaction product;
19: reaction product; 20: by-product oxide feed;
21: cyclone separator; 22: catalyst flow controller;
23: feeding port of the gaseous feedstock; 24: feeding pore;
25: top of the feeding pipeline; 26: reaction space;
27: reactor shell.

### DETAILED DESCRIPTION

The endpoints of ranges and any values disclosed herein are not limited to the precise range or value, but such ranges or values are to be understood to include values approximating such ranges or values. For numerical ranges, a combination can be made with each other between any two of the endpoints of ranges, between one of the endpoints of ranges and one individual point value, and between any two individual point values to obtain one or more new numerical ranges, which shall be deemed to be specifically disclosed herein.

In the present invention, when a technical solution is given in an open-ended form such as the form of “comprising”, “containing” or “including” certain listed elements, those skilled in the art will understand that the embodiments consisting of these elements or consisting essentially of these elements can obviously be used to implement the technical solution. Therefore, those skilled in the art will understand that the technical solutions given with the open-ended definitions in the present invention also cover specific embodiments that consists of the listed elements, or consists essentially of the listed elements.

Finally, unless otherwise specified, all percentages, parts, ratios, etc. mentioned in this specification are based on weight; however, when using weight as a basis does not conform to the common understanding of those skilled in the art, the basis is determined based on the common understanding of those skilled in the art.

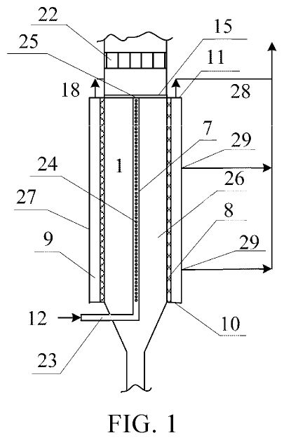

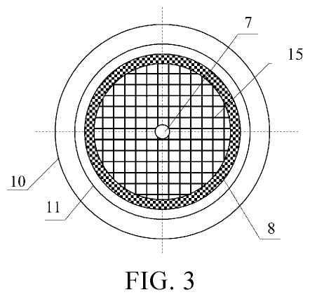

Referring to FIGS. 1 and 3, in order to achieve the aforementioned objects, the present invention provides for example an axial-radial short contact reactor 1, comprising the following components coaxially distributed from inside to outside:

a feeding pipeline 7, having a feeding port 23 at its lower end for introducing a gaseous feedstock, and allowing the feedstock to be transported from bottom to top, wherein feeding pores 24 are distributed on the feeding pipeline 7, so that the feedstock is transported outward from the feeding pipeline in a substantially radial direction, and preferably, the feeding pipeline 7 has a closed top 25 at its upper end;

an axial-radial reaction space 26, which is defined by the inner feeding pipeline 7 and an outer filter pipe wall 8, so that the feedstock transported radially outward contacts the catalyst transported axially from top to bottom in crossflow, and the gas-solid contact time is less than 3 seconds in the reaction space 26;

a filter pipe wall 8, which has a certain pore diameter so that the feedstock continues to be transported through pores outward and into a product gas channel 9, and the catalyst density in the product gas channel 9 is less than 10 kg/m³; and a reactor shell 27, which defines a product gas channel 9 together with the filter pipe wall 8, and has such a shape and structure that the residence time of a product gas in the product gas channel 9 is less than 15 seconds;

and the reactor 1 also has a catalyst distributor 15 arranged at its top, which distributor has a certain pore fraction, pore diameter and distribution thereof, so as to transport the catalyst by gravity from top to bottom, and allow the catalyst density in the reaction space 26 to reach the range from 80 kg/m³ to 400 kg/m³.

In an embodiment, the product gas is transported substantially from bottom to top in the product gas channel 9, and leaves the short contact reactor 1 from the product gas outlet 28 at the top of the product gas channel 9.

In the present invention, the expression “the feedstock is transported outward ‘in a substantially radial direction” means that during the process of the feedstock being transported outward from the feeding pipeline at the center of the reactor to the product gas channel, the main moving direction is radial, and during this process, its axial displacement does not exceed 50%, preferably 30% of its radial displacement.

In the present invention, the “ultra-short contact” reactor refers to a short gas-solid contact time between the gaseous feedstock and the catalyst, for example, less than 3 seconds.

The present invention configures the axial-radial short contact reactor through specific coordination between multiple components, which allows the gaseous feedstock transported radially outward to contact the catalyst transported axially from top to bottom in the axial-radial reaction space 26 in crossflow; and the so configured contact in crossflow achieves high catalyst bed density while achieving ultra-short contact time between the gaseous feedstock and the catalyst.

To this end, on one hand, according to an embodiment of the present invention, the feeding pipeline 7 has a pore fraction ranging from 5% to 20%. On the other hand, according to an embodiment of the present invention, the filter pipe wall 8 has an average pore diameter ranging from 5 microns to 30 microns.

According to the present invention, the pore fraction of the feeding pipeline 7 and the pore diameter of the filter pipe wall 8 allow the feedstock to achieve a gas-solid contact time of less than 2 seconds, e.g. from 0.1 seconds to 1.8 seconds in the reaction space 26.

According to the present invention, the pore diameter of the filter pipe wall 8 allows the amount of the catalyst transported outward through pores to be less than 1%, preferably less than 0.5%.

In another aspect, according to an embodiment of the present invention, the catalyst distributor 15 has such a pore fraction, pore diameter and distribution thereof that the reaction space 26 has a catalyst density of greater than 100 kg/m³, preferably greater than 150 kg/m³. To this end, preferably, the catalyst distributor 15 is a grille or a porous baffle with a pore fraction ranging from 60% to 95%.

Further, in order to adapt to the “short contact” between the gaseous feedstock and the catalyst, in the present invention, the reacted product gas which is sent into a product gas channel 9 and the possible unreacted part of gaseous feedstock are quickly removed through the product gas channel 9; for example, the shape and structure of the product gas channel 9 allows the residence time of the product gas in the product gas channel 9 to be less than 15 seconds, preferably less than 10 seconds.

Accordingly, the present invention provides an “uneven” configuration in at least some components of the reactor. In the present invention, the “uneven” configuration means that one or more structural features of the component are spatially non-uniform distribution.

On one hand, according to an embodiment of the “uneven” configuration of the present invention, the reactor shell 27 has an “uneven” outer diameter, e.g. a shape of a truncated cone, and a smaller diameter at its top, so that the diameter at the top 11 of the product gas channel 9 is smaller than the diameter at the bottom 10 of the product gas channel. For example, according to an embodiment of the “uneven” configuration of the present invention, the ratio of the diameter at the top 11 of the product gas channel to the diameter at the bottom 10 of the product gas channel ranges from 1:1.1 to 1:3, e.g. 1:1.8; and/or the reactor shell 27 has an angle between the side wall and the horizontal plane of from 60° to 90°.

Alternatively, the reactor shell 27 has an “even” outer diameter, but is equipped with “uneven” product gas withdrawn devices. For example, the lower part of the reactor shell 27 may be equipped with one or more additional product gas outlets 29. For example, in one embodiment, six product gas outlets 29 are evenly distributed in the lower part of the reactor shell 27, and the distance from the product gas outlet 29 to the bottom 10 of the gas channel comprises 15% of the total height of the gas channel 9.

Optionally, the reactor shell 27 has the above-mentioned “uneven” outer diameter, and is equipped with the above-mentioned “uneven” product gas withdrawn devices.

On the other hand, according to an embodiment of the “uneven” configuration of the present invention, the feeding pipeline 7 has a pore fraction that gradually increases from top to bottom. Preferably, the feeding pipeline 7 has a pore fraction in the upper region of from 5% to 10%, a pore fraction in the middle region of from 10% to 15%, and a pore fraction in the lower region of from 15% to 20%. In the present invention, the upper region, the middle region and the lower region of the feeding pipeline 7 refer to the division of each region at the respective positions approximately ⅓ of the height of the pipeline 7 in the height direction.

In another aspect, according to an embodiment of the “uneven” configuration of the present invention, the filter pipe wall 8 has an average pore diameter that gradually increases from top to bottom. For example, preferably, the filter pipe wall (8) has an average pore diameter in the upper region of from 5 microns to 10 microns, an average pore diameter in the middle region of from 10 microns to 20 microns, and an average pore diameter in the lower region of from 20 microns to 30 microns. In the present invention, the upper region, the middle region and the lower region of the filter pipe wall 8 refer to the division of each region at the respective positions approximately ⅓ of the height of the pipe wall 8 in the height direction.

The inventors have also surprisingly found that with the “uneven” configuration, it is beneficial to the distribution and flow of the product gas in the product gas channel 9, which is further beneficial to the smooth progress of the reaction in the reaction space 26. For example, without limitation to any known theory, for the process for preparing ethylene and propylene from methanol, the “uneven” configuration is beneficial to the ethylene and propylene selectivity.

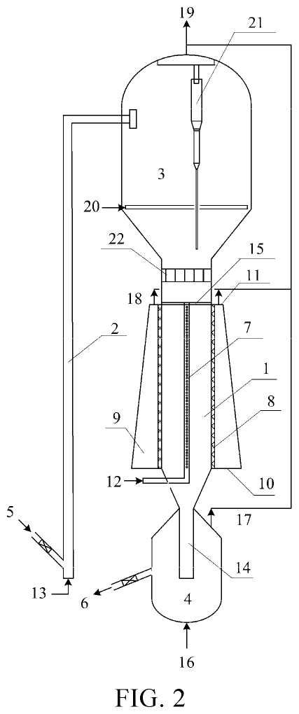

Correspondingly, based on the short contact reactor of the present invention, referring to FIG. 2, the present invention provides a short contact reaction system for preparing ethylene and propylene from methanol, comprising: an MTO short contact reactor 1 of the present invention, a riser reactor 2, a dense bed 3 and a stripper 4;
the MTO short contact reactor 1 is used to convert methanol to an olefin-rich product;
the riser reactor 2 is used to convert a mixed light hydrocarbon feed, including a mixture of C₄-C₆ non-aromatic hydrocarbons from the product of the MTO short contact reactor 1, into an olefin-rich product, which ascends into the dense bed 3;
the dense bed 3 is arranged above the short contact reactor 1, and is used to store and provide a catalyst required by the MTO short contact reactor 1 and convert a by-product oxide feed from the reaction product;
the stripper 4 is arranged below the short contact reactor 1 and is used to remove a reaction product entrained by the coked catalyst from the MTO short contact reactor 1.

By arranging the methanol feeding pipeline 7, the present invention can realize the contact of methanol with the catalyst in crossflow.

In an embodiment of the present invention, the diameter at the top 11 of the product gas channel is smaller than the diameter at the bottom 10 of the product gas channel, which can accelerate the ascending of the reaction product.

By providing the MTO short contact reactor 1 and the dense bed 3 at the same time, the present invention can achieve, after the ultra-short contact of double-olefins, the unconverted methanol treated in the dense bed 3 is enriched to obtain 20, thus greatly improving the conversion rate of methanol.

In the present invention, the product gas channel 9 is preferably arranged in an inclination direction, which can increase the ascending speed of the reaction product, thereby achieving rapid separation of the reaction product and the catalyst.

According to a preferable embodiment of the present invention, the filtering precision of the filter pipe wall 8 ranges from 10 microns to 30 microns. Using the filter pipe wall 8 of the present invention has the advantage of effectively intercepting the catalyst in the reaction product.

According to a preferable embodiment of the present invention, the ratio of the diameter at the top 11 of the product gas channel to the diameter at the bottom 10 of the product gas channel ranges from 1:1.1 to 1:3. Using the above configuration can increase the ascending speed of the reaction product, thereby achieving rapid separation of the reaction product and the catalyst.

According to a preferable embodiment of the present invention, a cyclone separator 21 is arranged in the dense bed 3. Arrangement of the cyclone separator is mainly used to separate the reaction product from the catalyst.

According to a preferable embodiment of the present invention, the reaction system further comprises a catalyst flow controller 22, which connects to the dense bed 3 and the MTO short contact reactor 1. Using the above configuration can effectively control the amount of the catalyst entering the MTO short contact reactor 1.

According to a preferable embodiment of the present invention, the reaction system further comprises a separation system, which is used to separate the reaction product 19 from the dense bed 3 and/or the MTO short contact reactor 1 into ethylene, propylene, and a mixture of C₄-C₆ non-aromatic hydrocarbons.

In the present invention, there is no special requirement to the catalyst flow controller, and any conventionally used catalyst flow controller can be used in the present invention. According to a preferable embodiment of the present invention, the catalyst flow controller 22 is for example a solid kicking device, a slide valve, and a plug valve.

In the present invention, there is no special requirement to the catalyst distributor 15, and any conventionally used catalyst distributor can be used in the present invention. For example, the catalyst distributor 15 is grille or a porous baffle, more preferably the catalyst distributor 15 has a pore fraction ranging from 60% to 95%.

According to a preferable embodiment of the present invention, the methanol feeding pipeline 7 is located in the center of the MTO short contact reactor 1. It is more preferred that feeding pores are evenly distributed on the methanol feeding pipeline 7, and it is further preferred that the pore fraction ranges from 5% to 20%.

Using the system of the present invention to carry out the reaction has the advantage of high ethylene and propylene selectivity. According to a preferred embodiment of the present invention, the present invention provides a process for producing ethylene and propylene from methanol through a short contact reaction, which process is carried out by using the reaction system of the present invention, and comprises:
a) rendering a methanol feedstock 12 to move upward through a methanol feeding pipeline 7 via a feeding port 23 of the gaseous feedstock, enter a reaction space 26 of an MTO short contact reactor 1 via a feeding port 24, and contact and react with a descending catalyst to produce a methanol reaction product 18 and a coked catalyst I; rendering the methanol reaction product 18 to enter a product gas channel 9 through filter pipe wall 8, leave the MTO short contact reactor and merge into the reaction product 19; rendering the coked catalyst to descend into a stripper 4 via a seal pipe 14;
b) rendering a mixed light hydrocarbon feed 13 and a steam to enter a riser reactor 2, contact and react with a regenerated catalyst 5, and ascend into a dense bed 3;
c) rendering a by-product oxide feed 20 to enter the dense bed 3, and contact and react with a catalyst to produce a reaction product and a coked catalyst II; rendering the coked catalyst II to enter the MTO short contact reactor 1 via a catalyst flow controller 22 and a catalyst distributor 15;
d) rendering a stripping medium 16 to enter a stripper 4 and contact the catalyst to perform stripping; rendering the resulting stripped product 17 to merge into the reaction product 19 and the resulting spent catalyst 6 to enter a regenerator for regeneration to produce a regenerated catalyst 5;
e) rendering the reaction product 19 to enter a subsequent separation system for separation to produce a mixed light hydrocarbon feed 13 and a by-product oxide feed 20.

According to a preferable embodiment of the present invention, the mixed light hydrocarbon feed 13 at least includes a mixture of C₄-C₆ non-aromatic hydrocarbons obtained from the separation system.

According to a preferable embodiment of the present invention, by-product oxide feed 20 is composed of a by-product mixed oxide and water as generated from the reaction, preferably wherein the mixed oxide is comprised in an amount ranging from 5% to 80%, the mixed oxide contains methanol and at least one of ethanol, propanol, butanol, ethanal, propanal, butanal, acetone, butanone, formic acid, acetic acid, and propionic acid, the aldehydes and ketones are comprised in an amount ranging from 30% to 60% in the mixed oxide, and methanol is comprised in an amount ranging from 0.01% to 30% in the mixed oxide.

According to a preferable embodiment of the present invention, the operation conditions in the MTO short contact reactor 1 includes:
the catalyst temperature: 450-500° C.,
the reaction gauge pressure: 0.01-0.3 MPa,
the mass space velocity of methanol: 2-15 h⁻¹, and
the catalyst density: 100-400 kg/m³.

According to a preferable embodiment of the present invention, the operation conditions in the riser reactor 2 includes:
the catalyst temperature: 530-650° C.,
the gas linear velocity: 1.1-15 m/s,
the mass space velocity of the mixed light hydrocarbon feed 13: 5-30 h⁻¹, and
the catalyst density: 20-100 kg/m³.

According to a preferable embodiment of the present invention, the operation conditions in the dense bed 3:
the catalyst temperature: 480-580° C.,
the gas linear velocity: 0.3-1 m/s,
the mass space velocity of the by-product oxide feed 20: 0.3-3 h⁻¹,
the catalyst density: 180-400 kg/m³.

According to a preferable embodiment of the present invention, the weight ratio of the mixed light hydrocarbon feed 13 to the steam ranges from 1: 0.5 to 1:3.

In the present invention, the selection range of the catalyst types is relatively wide, and all conventionally used catalysts for producing propylene and ethylene from methanol can be used in the present invention. According to a preferable embodiment of the present invention, the catalyst is SAPO-34 molecular sieve catalyst.

According to a preferable embodiment of the present invention, the regenerated catalyst 5, based on the total weight of the catalyst, has a carbon content of less than 0.1%.

According to a preferable embodiment of the present invention, the stripping medium 16 can be the conventionally used stripping medium, for example one or more of steam and inert gases. According to the present invention, the preferable stripping medium is steam.

According to a preferable embodiment of the present invention, the short contact reaction system for preparing ethylene and propylene from methanol of the present invention comprises an MTO short contact reactor 1, a riser reactor 2, a dense bed 3, and a stripper 4; wherein the MTO short contact reactor 1 consists of a methanol feeding pipeline 7, a filter pipe wall 8, a product gas channel 9 and a catalyst distributor 15; the methanol feeding pipeline 7, the filter pipe wall 8 and the product gas channel 9 are coaxially arranged; the methanol feeding pipeline 7, the filter pipe wall 8 and the product gas channel 9 are located in turn from the inside to the outside; a catalyst distributor 15 is located at the top of the MTO ultra-short reactor 1, a seal pipe 14 is connected to the bottom of the MTO ultra-short reactor 1; the seal pipe 14 is located in the stripper 4; the diameter at the top 11 of the product gas channel is smaller than that at the bottom 10 of the product gas channel; a cyclone separator 21 is located in the dense bed 3; a catalyst flow controller 22 connects to the dense bed 3 and the MTO short contact reactor 1.

According to a preferable embodiment of the present invention, the process comprises the following steps:
rendering a methanol feedstock 12 to enter an MTO short contact reactor 1 via a methanol feeding pipeline 7, and contact and react with a descending catalyst to produce a methanol reaction product 18 and a coked catalyst I; rendering the methanol reaction product 18 to enter a product gas channel 9 via a filter pipe wall 8, leave the MTO short contact reactor 1 and merge into the reaction product 19; rendering the coked catalyst to descend into a stripper 4 via a seal pipe 14;
rendering a mixed light hydrocarbon feed 13 and a part of steam to enter a riser reactor 2, contact and react with a regenerated catalyst 5, and ascend into a dense bed 3;
rendering a by-product oxide feed 20 to enter the dense bed 3, and contact and react with the catalyst to produce a reaction product and a coked catalyst II; rendering the coked catalyst II to enter the MTO short contact reactor 1 via a catalyst flow controller 22 and a catalyst distributor 15;
rendering a stripping medium 16 to enter a stripper 4 and contact the catalyst to perform stripping;
rendering the resulting stripped product 17 to merge into the reaction product 19 and the resulting spent catalyst 6 to enter a regenerator for regeneration to produce a regenerated catalyst 5;
rendering the reaction product 19 to enter a subsequent separation system for separation to produce a mixed light hydrocarbon feed 13 and a by-product oxide feed 20.

According to the present invention, preferably, the mixed light hydrocarbon feed 13 at least includes a mixture of C₄-C₆ non-aromatic hydrocarbons obtained from the separation system.

According to the present invention, preferably, the by-product oxide feed 20 is composed of a by-product mixed oxide and water as generated from the reaction, wherein the mixed oxide is comprised in an amount ranging from 5% to 80%, the mixed oxide contains methanol and at least one of ethanol, propanol, butanol, ethanal, propanal, butanal, acetone, butanone, formic acid, acetic acid, propionic acid, wherein the aldehydes and ketones is comprised in an amount ranging from 30% to 60% in the mixed oxide, and methanol is comprised in an amount ranging from 0.01% to 30% in the mixed oxide.

According to the present invention, preferably, in the MTO short contact reactor 1, the catalyst temperature ranges from 450° C. to 500° C., the reaction gauge pressure ranges from 0.01 MPa to 0.3 MPa, the mass space velocity of methanol ranges from 2 h⁻¹ to 15 h⁻¹, and the catalyst density ranges from 100 kg/m³ to 400 kg/m³.

According to the present invention, preferably, in the riser reactor 2, the catalyst temperature ranges from 530° C. to 650° C., the gas linear velocity ranges from 1.1 m/s to 15 m/s, the mass space velocity of the mixed light hydrocarbon feed 13 ranges from 5 h⁻¹ to 30 h⁻¹, and the catalyst density ranges from 20 kg/m³ to 100 kg/m³.

According to the present invention, preferably, in the dense bed 3, the catalyst temperature ranges from 480° C. to 580° C., the gas linear velocity ranges from 0.3 m/s to 1 m/s, the mass space velocity of the by-product oxide feed 20 ranges from 0.3 h⁻¹ to 3 h⁻¹, and the catalyst density ranges from 180 kg/m³ to 400 kg/m³.

According to the present invention, preferably, the weight ratio of the mixed light hydrocarbon feed 13 to the steam ranges from 1:0.5 to 1:3.

According to the present invention, preferably, the catalyst is SAPO-34 molecular sieve catalyst.

According to the present invention, preferably, the regenerated catalyst 5, based on the total weight of the catalyst, has a carbon content of less than 0.1%.

In the above-mentioned technical solution, preferably, the stripping medium 16 is steam and/or nitrogen gas.

The present invention will be further described below through examples, but it is not limited to these examples.

### Example 1

An apparatus as shown in FIG. 2 was used, and comprised an MTO short contact reactor 1, a riser reactor 2, a dense bed 3 and a stripper 4; wherein, the MTO short contact reactor 1 consisted of a methanol feeding pipeline 7, a filter pipe wall 8, a product gas channel 9 and a catalyst distributor 15; the methanol feeding pipeline 7, the filter pipe wall 8 and the product gas channel 9 were coaxially arranged; the methanol feeding pipeline 7, the filter pipe wall 8 and the product gas channel 9 were located in turn from the inside to the outside; a catalyst distributor 15 was located at the top of the MTO ultra-short reactor 1, a seal pipe 14 was connected to the bottom of the MTO ultra-short reactor 1; the seal pipe14 was located in the stripper 4; the diameter at the top 11 of the product gas channel was smaller than that at the bottom 10 of the product gas channel; a cyclone separator 21 was located in the dense bed 3; a catalyst flow controller 22 was connected to the dense bed 3 and the MTO short contact reactor 1.

A methanol feedstock 12 entered an MTO short contact reactor 1 via a methanol feeding pipeline 7, and contacted and reacted with a descending catalyst to produce a methanol reaction product 18 and a coked catalyst I; the methanol reaction product 18 entered a product gas channel 9 via a filter pipe wall 8, left the MTO short contact reactor 1 and merged into the reaction product 19; the coked catalyst descended into a stripper 4 via a seal pipe 14; a mixed light hydrocarbon feed 13 and a part of steam entered a riser reactor 2, contacted and reacted with a regenerated catalyst 5, and ascended into a dense bed 3; a by-product oxide feed 20 entered the dense bed 3, and contacted and reacted with the catalyst to produce a reaction product and a coked catalyst II, the coked catalyst II entered the MTO short contact reactor 1 via a catalyst flow controller 22 and a catalyst distributor 15; a stripping medium 16 entered a stripper 4 and contacted the catalyst to perform stripping, the resulting stripped product 17 merged into the reaction product 19, and the resulting spent catalyst 6 entered a regenerator for regeneration to produce a regenerated catalyst 5; the reaction product 19 entered a subsequent separation system for separation to produce a mixed light hydrocarbon feed 13 and a by-product oxide feed 20.

The filter pipe wall 8 had an average pore diameter in the upper region of 5 microns, an average pore diameter in the middle region of 10 microns, and an average pore diameter in the lower region of 20 microns.

The ratio of the diameter at the top 11 of the product gas channel to the diameter at the bottom 10 at the product gas channel was 1:1.1, the reactor shell 27 had an angle between the side wall and the horizontal plane of 85°.

The catalyst distributor 15 was a grille with a pore fraction of 60%.

The methanol feeding pipeline 7 was located in the center of the MTO short contact reactor 1.

The methanol feeding pipeline 7 had a pore fraction in the upper region of 5%, a pore fraction in the middle region of 10%, and a pore fraction in the lower region of 15%.

The mixed light hydrocarbon feed 13 at least included a mixture of C₄-C₆ non-aromatic hydrocarbons obtained from the separation system.

The by-product oxide feed 20 was composed of a by-product mixed oxide and water as generated from the reaction, wherein the mixed oxide was comprised in an amount of 45%. The mixed oxide contained methanol and at least one of ethanol, propanol, butanol, ethanal, propanal, butanal, acetone, butanone, formic acid, acetic acid, and propionic acid, the aldehydes and ketones were comprised in an amount of 50% in the mixed oxide, and methanol was comprised in an amount of 25% in the mixed oxide.

In the MTO short contact reactor 1, the catalyst temperature was 480° C., the reaction gauge pressure was 0.1 MPa, the mass space velocity of methanol was 10 h⁻¹, and the catalyst density was 200 kg/m³.

In the riser reactor 2, the catalyst temperature was 600° C., the gas linear velocity was 5 m/s, the mass space velocity of the mixed light hydrocarbon feed 13 was 18 h⁻¹, and the catalyst density was 50 kg/m³.

In the dense bed 3, the catalyst temperature was 500° C., the gas linear velocity was 0.5 m/s, the mass space velocity of the by-product oxide feed 20 was 1 h⁻¹, and the catalyst density was 350 kg/m³.

The weight ratio of the mixed light hydrocarbon feed 13 to the steam was 1:1.

The catalyst was SAPO-34 molecular sieve catalyst.

The regenerated catalyst 5, based on the total weight of the catalyst, had a carbon content of 0.02%.

The stripping medium 16 was steam.

The result showed that the ethylene and propylene carbon-based selectivity reached 92.5 wt. %.

### Example 2

The apparatus and conditions of Example 1 were used except that the filtering precision of the filter pipe wall 8 was 30 microns.

The ratio of the diameter at the top 11 of the product gas channel to the diameter at the bottom of the product gas channel was 1:3, the reactor shell 27 had an angle between the side wall and the horizontal plane of 60°.

The catalyst distributor 15 was a grille with the pore fraction of 95%.

The filter pipe wall 8 had an average pore diameter in the upper region of 10 microns, an average pore diameter in the middle region of 20 microns, and an average pore diameter in the lower region of 30 microns.

The methanol feeding pipeline 7 was located in the center of the MTO short contact reactor 1. The methanol feeding pipeline 7 had a pore fraction in the upper region of 10%, a pore fraction in the middle region of 15%, and a pore fraction in the lower region of 20%.

The result showed that the ethylene and propylene carbon-based selectivity reached 91.3 wt. %.

### Example 3

The apparatus and conditions of Example 1 were used except that the filtering precision of the filter pipe wall 8 was 20 microns.

The ratio of the diameter at the top 11 of the product gas channel to the diameter at the bottom 10 of the product gas channel was 1:1.9, the reactor shell 27 had an angle between the side wall and the horizontal plane of 70°.

The catalyst distributor 15 was a grille with the pore fraction of 75%.

The methanol feeding pipeline 7 was located in the center of the MTO short contact reactor 1. The methanol feeding pipeline 7 had a pore fraction in the upper region of 7%, a pore fraction in the middle region of 12%, and a pore fraction in the lower region of 18%.

The filter pipe wall 8 had an average pore diameter in the upper region of 7 microns, an average pore diameter in the middle region of 15 microns, and an average pore diameter in the lower region of 25 microns.

The result showed that the ethylene and propylene carbon-based selectivity reached 93.0 wt. %.

### Example 4

The apparatus of Example 3 was used except that in the MTO short contact reactor 1, the catalyst temperature was 450° C., the reaction gauge pressure was 0.01 MPa, the mass space velocity of methanol was 2 h⁻¹, and the catalyst density was 100 kg/m³.

In the riser reactor 2, the catalyst temperature was 530° C., the gas linear velocity was 1.1 m/s, the mass space velocity of the mixed light hydrocarbon feed 13 was 5 h⁻¹, and the catalyst density was 20 kg/m³.

In the dense bed 3, the catalyst temperature was 480° C., the gas linear velocity was 0.3 m/s, the mass space velocity of the by-product oxide feed 20 was 0.3 h⁻¹, and the catalyst density was 180 kg/m³.

The weight ratio of the mixed light hydrocarbon feed 13 to the steam was 1:0.5.

The catalyst was SAPO-34 molecular sieve catalyst.

The regenerated catalyst 5, based on the total weight of the catalyst, had a carbon content of 0.09%.

The stripping medium 16 was steam.

The result showed that the ethylene and propylene carbon-based selectivity reached 91.4 wt. %.

### Example 5

The apparatus of Example 3 was used except that in the MTO short contact reactor 1, the catalyst temperature was 500° C., the reaction gauge pressure was 0.3 MPa, the mass space velocity of methanol was 15 h⁻¹, and the catalyst density was 400 kg/m³.

In the riser reactor 2, the catalyst temperature was 650° C., the gas linear velocity was 15 m/s, the mass space velocity of the mixed light hydrocarbon feed 13 was 30 h⁻¹, and the catalyst density was 100 kg/m³.

In the dense bed 3, the catalyst temperature was 580° C., the gas linear velocity was 1 m/s, the mass space velocity of the by-product oxide feed 20 was 3 h⁻¹, and the catalyst density was 400 kg/m³.

The weight ratio of the mixed light hydrocarbon feed 13 to the steam was 1:3.

The catalyst was SAPO-34 molecular sieve catalyst.

The regenerated catalyst 5, based on the total weight of the catalyst, had a carbon content of 0.01%.

The stripping medium 16 was steam.

The result showed that the ethylene and propylene carbon-based selectivity reached 93.1 wt. %.

### Example 6

The apparatus of Example 3 was used except that in the MTO short contact reactor 1, the catalyst temperature was 490° C., the reaction gauge pressure was 0.18 MPa, the mass space velocity of methanol was 13 h⁻¹, and the catalyst density was 300 kg/m³.

In the riser reactor 2, the catalyst temperature was 580° C., the gas linear velocity was 10 m/s, the mass space velocity of the mixed light hydrocarbon feed 13 was 23 h⁻¹, and the catalyst density was 35 kg/m³.

In the dense bed 3, the catalyst temperature was 520° C., the gas linear velocity was 0.8 m/s, the mass space velocity of the by-product oxide feed 20 was 1.8 h⁻¹, and the catalyst density was 300 kg/m³.

The weight ratio of the mixed light hydrocarbon feed 13 to the steam was 1:2.

The catalyst was SAPO-34 molecular sieve catalyst.

The regenerated catalyst 5, based on the total weight of the catalyst, had a carbon content of 0.03%.

The stripping medium 16 was nitrogen gas.

The result showed that the ethylene and propylene carbon-based selectivity reached 94.0 wt. %.

### Comparative Example 1

The apparatus and conditions of Example 6 were used except that the feeding amount of the mixed light hydrocarbon feed 13 was zero, and steam was introduced into the riser reactor 2. The result showed that the ethylene and propylene carbon-based selectivity reached 85.2 wt. %.

### Comparative Example 2

The apparatus and conditions of Example 6 were used except that the feeding amount of the by-product oxide feed 20 was 0.

The result showed that the ethylene and propylene carbon-based selectivity reached 88.4 wt. %.

### Comparative Example 3

The apparatus and conditions of Example 6 were used except that the ratio of the diameter at the top 11 of the product gas channel to the diameter at the bottom 10 of the product gas channel was 1:1.

The result showed that the ethylene and propylene carbon-based selectivity reached 89.0 wt. %.

### Example 7

The apparatus of Example 6 was used except that in the MTO short contact reactor 1, the catalyst temperature was 520° C., the reaction gauge pressure was 0.4 MPa, the mass space velocity of methanol was 20 h⁻¹, and the catalyst density was 450 kg/m³.

The result showed that the ethylene and propylene carbon-based selectivity reached 90.3 wt. %. The preferred embodiments of the present invention have been described in detail above, but the present invention is not limited thereto. Within the scope of the technical concept of the present invention, a variety of simple modifications can be made to the technical solutions of the present invention, including the combinations of various technical features in any other suitable manners. These simple modifications and combinations should also be regarded as the disclosures content of the present invention, and all belong to the protection scope of the present invention.

1. An axial-radial short contact reactor (1), comprising the following components coaxially distributed from inside to outside:
a feeding pipeline (7), having a feeding port (23) at its lower end for introducing a gaseous feedstock, and allowing the feedstock to be transported from bottom to top, wherein feeding pores (24) are distributed on the feeding pipeline (7), so that the feedstock is transported outward from the feeding pipeline in a substantially radial direction, and preferably, the feeding pipeline (7) has a closed top (25) at its upper end;
an axial-radial reaction space (26), which is defined by the inner feeding pipeline (7) and an outer filter pipe wall (8), so that the feedstock transported radially outward contacts the catalyst transported axially from top to bottom in crossflow, and the gas-solid contact time is less than 3 seconds in the reaction space (26);
a filter pipe wall (8), which has a certain pore diameter so that the feedstock continues to be transported through pores outward and into a product gas channel (9), and the catalyst density in the product gas channel (9) is less than 10 kg/m³; and
a reactor shell (27), which defines a product gas channel (9) together with the filter pipe wall (8), and has such a shape and structure that the residence time of a product gas in the product gas channel (9) is less than 15 seconds;
and the reactor (1) also has a catalyst distributor (15) arranged at its top, which distributor has a certain pore fraction, so as to transport the catalyst by gravity from top to bottom, and allow the catalyst density in the reaction space (26) to reach the range from 80 kg/m³ to 400 kg/m³.

2. The reactor according to claim 1, wherein the pore fraction of the feeding pipeline (7) and the pore diameter of the filter pipe wall (8) allow the feedstock to achieve a gas-solid contact time of less than 2 seconds, e.g. from 0.1 seconds to 1.8 seconds in the reaction space (26).

3. The reactor according to claim 1, wherein the feeding pipeline (7) has a pore fraction that gradually increases from top to bottom; preferably, the feeding pipeline (7) has a pore fraction in the upper region of from 5% to 10%, a pore fraction in the middle region of from 10% to 15%, and a pore fraction in the lower region of from 15 to 20%.

4. The reactor according to claim 1, wherein the filter pipe wall (8) has an average pore diameter ranging from 5 microns to 30 microns.

5. The reactor according to claim 1, wherein the filter pipe wall (8) has an average pore diameter that gradually increases from top to bottom; preferably, the filter pipe wall (8) has an average pore diameter in the upper region of from 5 microns to 10 microns, an average pore diameter in the middle region of from 10 microns to 20 microns, and an average pore diameter in the lower region of from 20 microns to 30 microns.

6. The reactor according to claim 1, wherein the catalyst distributor (15) is configured so that the reaction space (26) has a catalyst density of greater than 100 kg/m³, preferably greater than 150 kg/m³; preferably, the catalyst distributor (15) is a grille or porous baffle with a pore fraction ranging from 60% to 95%.

7. The reactor according to claim 1, wherein, the reactor shell (27) has a shape of a truncated cone, and a smaller diameter at its top, so that the diameter at the top (11) of the product gas channel (9) is smaller than the diameter at the bottom (10) of the product gas channel.

8. The reactor according to claim 7, wherein the ratio of the diameter at the top (11) of the product gas channel to the diameter at the bottom (10) of the product gas channel ranges from 1:1.1 to 1:3; and/or the reactor shell (27) has an angle between the side wall and the horizontal plane of from 60° to 85°.

9. The reactor according to claim 1, wherein, the feeding pipeline (7) has a pore fraction ranging from 5% to 20%.

10. A short contact reaction system for preparing ethylene and propylene from methanol, comprising: a short contact reactor (1) according to claim 1, a riser reactor (2), a dense bed (3), and a stripper (4);
the MTO short contact reactor (1) is used to convert methanol to an olefin-rich product;
the riser reactor (2) is used to convert a mixed light hydrocarbon feed, including a mixture of C₄-C₆ non-aromatic hydrocarbons from the product of the MTO short contact reactor (1), into an olefin-rich product, which ascends into the dense bed (3);
the dense bed (3) is arranged above the short contact reactor (1), and is used to store and provide a catalyst required by the short contact reactor (1) and convert a by-product oxide feed from the reaction product;
the stripper (4) is arranged below the short contact reactor (1) and is used to remove a reaction product entrained by the coked catalyst from the MTO short contact reactor (1).

11. The reaction system according to claim 10, wherein
a cyclone separator (21) is arranged in the dense bed (3); and/or
the reaction system further comprises a catalyst flow controller (22), which connects to the dense bed (3) and the MTO short contact reactor (1); and/or
the reaction system further comprises a separation system, which is used to separate the reaction product (19) from the dense bed (3) and/or the MTO short contact reactor (1) into ethylene, propylene, and a mixture of C₄-C₆ non-aromatic hydrocarbons.

12. The reaction system according to claim 11, wherein
the catalyst flow controller (22) is a solid kicking device, a slide valve or a plug valve.

13. A process for producing ethylene and propylene from methanol through a short contact reaction, characterized in that said process is performed using the reaction system according to claim 10, and comprises:
a) rendering a methanol feedstock (12) to enter an MTO short contact reactor (1) via a methanol feeding pipeline (7), and contact and react with a descending catalyst to produce a methanol reaction product (18) and a coked catalyst I; rendering the methanol reaction product (18) to enter a product gas channel (9) via a filter pipe wall (8), leave the MTO short contact reactor (1) and merge into a reaction product (19); rendering the coked catalyst to descend into a stripper (4) via a seal pipe (14);
b) rendering a mixed light hydrocarbon feed (13) and a steam to enter a riser reactor (2), contact and react with a regenerated catalyst (5), and ascend into a dense bed (3);
c) rendering a by-product oxide feed (20) to enter the dense bed (3), and contact and react with a catalyst to produce a reaction product and a coked catalyst II; rendering the coked catalyst II to enter the MTO short contact reactor (1) via a catalyst flow controller (22) and a catalyst distributor (15);
d) rendering a stripping medium (16) to enter a stripper (4) and contact the catalyst to perform stripping; rendering the resulting stripped product (17) to merge into the reaction product (19) and the resulting spent catalyst (6) to enter a regenerator for regeneration to produce a regenerated catalyst (5);
e) rendering the reaction product (19) to enter a subsequent separation system for separation to produce a mixed light hydrocarbon feed (13) and a by-product oxide feed (20).

14. The process according to claim 13, wherein
the mixed light hydrocarbon feed (13) at least includes a mixture of C₄-C₆ non-aromatic hydrocarbons obtained from the separation system; and/or
the by-product oxide feed (20) is composed of a by-product mixed oxide and water as generated from the reaction,
wherein the mixed oxide is comprised in an amount ranging from 5% to 80%, the mixed oxide contains methanol and at least one of ethanol, propanol, butanol, ethanal, propanal, butanal, acetone, butanone, formic acid, acetic acid, and propionic acid, wherein the aldehydes and ketones are comprised in an amount ranging from 30% to 60% in the mixed oxide, and methanol is comprised in an amount ranging from 0.01% to 30% in the mixed oxide.

15. The process according to claim 13, wherein
the operation conditions in the MTO short contact reactor (1) includes:
the catalyst temperature: 450-500° C.,
the reaction gauge pressure: 0.01-0.3 MPa,
the mass space velocity of methanol: 2-15 h⁻¹,
the catalyst density: 100-400 kg/m³; and/or
the operation conditions in the riser reactor (2) includes:
the catalyst temperature: 530-650° C.,
the gas linear velocity: 1.1-15 m/s,
the mass space velocity of the mixed light hydrocarbon feed (13): 5-30 h⁻¹,
the catalyst density: 20-100 kg/m³; and/or
the operation conditions in the dense bed (3) includes:
the catalyst temperature: 480-580° C.,
the gas linear velocity: 0.3-1 m/s,
the mass space velocity of the by-product oxide feed (20): 0.3-3 h⁻¹,
the catalyst density: 180-400 kg/m³.

16. The process according to claim 13, wherein
the weight ratio of the mixed light hydrocarbon feed (13) to the steam ranges from 1:0.5 to 1:3; and/or
the catalyst is SAPO-34 molecular sieve catalyst; and/or
the regenerated catalyst (5), based on the total weight of the catalyst, has a carbon content of less than 0.1%; and/or
the stripping medium (16) is one or more of steam and inert gases.
